(19) United States
(12) Patent Application Publication
(10) Pub. No.: US 2017/0032296 A1
(43) Pub. Date: Feb. 2, 2017

(54) ULTRASOUND RESOURCE MANAGEMENT SYSTEM

(71) Applicant: Koninklijke Philips N.V., Eindhoven (NL)

(72) Inventors: Paul Richard SIMONS, Cambridge (GB); Robert FOWLER, Bristol (GB)

(21) Appl. No.: 15/303,272

(22) PCT Filed: Apr. 15, 2015
(86) PCT No.: PCT/EP2015/058116

§ 371 Date: Oct. 11, 2016

(30) Foreign Application Priority Data
Apr. 24, 2014 (EP) 14165799.9

(51) Int. Cl. G06Q 10/06; G06Q 30/04

(52) U.S. Cl. CPC G06Q 10/0633; G06Q 30/04

(57) ABSTRACT

A server comprising: a transceiver configured to receive from at least one ultrasound machine at least one data log comprising at least one data record based on the operation of the ultrasound machine; a record parser configured to process the at least one data log received from the at least one ultrasound machine before storing the at least one data log in a database; a record analyser configured to analyse the at least one data log to determine at least one metric associated with the at least one ultrasound machine; a report generator configured to generate a report for a client based on applying the at least one metric to a utilization/billing model.

### FIELD OF THE INVENTION

This invention relates to ultrasound resource management systems, and specifically but not limited to cost related ultrasound resource management systems.

### BACKGROUND OF THE INVENTION

Diagnostic sonography (ultrasonography) is an ultrasound-based diagnostic imaging technique used for visualizing subcutaneous body structures including tendons, muscles, joints, vessels and internal organs for possible pathology or lesions.

Ultrasound images (sonograms) are made by sending a pulse of ultrasound into tissue using an ultrasound transducer (probe), part of an ultrasound machine. The sound reflects and echoes off parts of the tissue, the echo is recorded and displayed as an image to the operator typically by a further part of the ultrasound machine.

Compared to other prominent methods of medical imaging, ultrasonography performed by ultrasound machines has several advantages. It provides images in real-time (rather than after an acquisition or processing delay). Furthermore the ultrasound machines are typically portable and can be brought to a sick patient's bedside. The ultrasound machines are also typically substantially lower in cost than other diagnostic machines.

Ultrasound machines come at a wide range of different price points from 30,000-200,000 Euros. Ultrasound machines furthermore make use of disposables, and peripherals.

As they are seen as being a relatively inexpensive diagnostic tool, their greater number and their mobility can lead to issues with respect to determining whether the ultrasound machines in operation are being used efficiently and in a cost effective manner. Similarly the many different types of business that need them can require different pay-per-use models for ultrasound machines in order to more efficiently control their use. For example some ultrasound machines (such as those operating in the UK National Health Service hospitals) are in near continuous use, whereas other machines (such as those operating in private hospitals) are used sparingly and could be more efficiently managed. Similarly there are also new enterprises like Any qualifying Provider (AQP) such as General Practitioners (GPs) and research hospitals which would benefit from the ability to operate an ultrasound machine but may not be able to afford the capital required to purchase one, and furthermore even where they could afford the capital need to efficiently and cost effectively operate any machine (for example implement a different methods to pay for the ultrasound machine if they are to afford the equipment).

As the various operators usage pattern of an ultrasound machine differ their utilization optimisation methods will differ. Furthermore where the ultrasound machine is operated on a pay-per-use or resource operation cost basis then the pay-per-use or resource operation cost model or method also needs to be different. In such a manner where the utilization or cost operation can be made more efficient it would enable improved ultrasound machine workflow, increased profits, reduced risks associated with capital equipment costs and increased desirability of the product.

For example increasingly GPs want to provide ultrasound services to their patients, for example for pregnancy, breast and abdominal scans and as such have been using AQPs to provide such services. These AQPs could be single agents or groups that can charge per scan. However, setup costs for these organisations are expensive (equipment, facilities, rooms, booking systems etc.). So where the ultrasound machines can be efficiently utilized and/or the costs linked to the reimbursement rate, a pay-per-use model becomes a very attractive option for the AQPs. The AQP model is comparable with business needs in the private sector and therefore is applicable to global markets as in other countries there is more private that public health.

Philips MediGo is a known pay per use (PPU) system and provides access to Philips medical technology on a fee basis, with predefined minimum and maximum patient volumes. A typical PPU model is based on calculating a monthly base price for the equipment based on typical volume levels.

### SUMMARY OF THE INVENTION

The invention is defined by the claims.

According to some embodiments there may be provided an ultrasound machine comprising: a utilization determiner configured to determine at least one utilization data record based on the operation of the ultrasound machine; a data compiler configured to generate a data log comprising the at least one utilization data record; a transmitter configured to transmit the data log to a server configured to analyse the data log of the ultrasound machine.

Thus according to some embodiments the ultrasound machine can generate suitable utilization data which may be used to more efficiently (or more cost effectively) operate the ultrasound machine or other ultrasound machines.

The ultrasound machine may further comprise at least one clock coupled to the utilization determiner, wherein the at least one utilization data record comprises at least one clock data field.

Thus in such embodiments the ultrasound machine utilization data may include timing or clock data within it to enable a more efficient or more cost efficient use of the ultrasound machine or other ultrasound machines being analysed.

The ultrasound machine may further comprise a mode determiner configured to determine the operational mode of the ultrasound machine, the mode determiner being further coupled to the utilization determiner, wherein the at least one utilization data record comprises at least one operational mode data field.

In such an arrangement the ultrasound machine utilization data may include information as to the mode of operation of the machine to enable a more efficient or more cost efficient use of the ultrasound machine or other ultrasound machines being analysed.

The ultrasound machine may further comprise a location determiner configured to determine the location of the ultrasound machine, the location determiner being further coupled to the utilization determiner, wherein the at least one utilization data record comprises at least one location data field.

Thus in some embodiments the ultrasound machine utilization data may include location data to enable a more efficient or more cost efficient use of the ultrasound machine or other ultrasound machines in the same area which are also analysed.

The ultrasound machine may further comprise a user interface configured to receive at least one of: an ultrasound operator identifier; an organization identifier; and a location identifier, the user interface may be configured to be further coupled to the utilization determiner, wherein the at least one utilization data record may comprise at least one of: the ultrasound operator identifier; the organization identifier; and the location identifier when received.

Thus in some embodiments the ultrasound machine utilization data may include data about the operator, such as the operator or referrer, the organization using the ultrasound, such as the hospital, hospital department or GP practice, and the location of the operator to enable a more efficient or more cost efficient use of the ultrasound machine or other ultrasound machines (in the same area) which are also analysed.

According to a first aspect there is provided a server comprising: a transceiver configured to receive from at least one ultrasound machine at least one data log comprising at least one data record based on the operation of the ultrasound machine; a record parser configured to process the at least one data log received from the at least one ultrasound machine before storing the at least one data log in a database; a record analyser configured to analyse the at least one data log to determine at least one metric associated with the at least one ultrasound machine; a report generator configured to generate a report for a client based on applying the at least one metric to a utilization/billing model.

The report generator may comprise a billing report generator configured to generate a billing report for a client based on applying the at least one metric to the billing model.

The billing model may be a pay-per-use billing model.

The billing report generator configured to generate a billing report for a client based on applying the at least one metric to a billing model may be configured to generate the billing report based on at least one of:

a charged fixed rate over the length of a contract; a charge based on the initial price and the estimated devaluation of the ultrasound machine due to aging of the technology and/or parts; a charge based on a combination of average duration and number of scans; a charge based on the volume of patients; a charged percentage of national reimbursement tariff for ultrasound scans; a percentage of costs charged to the customer; a charge based on a number of prepaid hours per month at a fixed rate where extra hours have additional cost; a charge based on costs of disposables that are used in procedures; a charge based on a named sonographer; a charge based on a named clinicians; a charge based on a timed rate; a charge for each transducer in use;

a charge for each preset of the ultrasound machine in use; a charge based on when the ultrasound machine is powered on; a charge based on the ultrasound machine is used for patient studies;

a charge based on the efficiency of usage of the ultrasound machine;

a charge based on a number, type and/or length of each study performed by the ultrasound machine;

a charge based on the type of department that the scan is required; a charge based on the location of the ultrasound machine; a charge based on the intern& address of the ultrasound machine; a charge based on the time of day; and a charge based on the day of the week.

The record parser/processor may be configured to anonymise named data within the at least one data log before the data is stored on the database.

Thus in some embodiments a name is associated with an ID label or number in such a way that they are traceable as an individual but not named.

In some embodiments the server is able to receive the ultrasound machine log data from at least one and in some embodiments several ultrasound machines which are linked in some manner but not necessarily owned or loaned to a single operator. The server may then in such embodiments perform processing on the data to enable storage of the data and furthermore analysis on the ultrasound machine log data to enable the determination of at least one metric, which may then be applied to suitable utilization/billing model in order to generate a report for a client. This report may in some embodiments form the basis of a suitable efficient control of the ultrasound machines or enable a more cost efficient operation of the ultrasound to be determined based on the billing report.

The server may further comprise a request determiner configured to receive at least one request from a client, the request comprising at least one of: a report type identifier; a utilization/billing model identifier; a data log sub-set identifier; a client identifier; a client organization identifier; and a client location identifier.

Thus in such embodiments the report may be generated based on a report request from a client, such as where a client wished to determine whether the ultrasound machine is being efficiently or cost efficiently used. Furthermore the request may enable a report to be generated based on an identifier such that a type of report, such as billing or utilization efficiency report can be generated, or the report based on a sub-set of the data log entries, such as a particular type of machine or regional report is generated.

The server may be further configured to generate a report based on applying the at least one metric to the utilization/billing model associated with the utilization/billing model identifier. In such embodiments the report requested may be one generated based on the utilization model or the billing model which is applied to the client or a utilization or billing model the client wishes to examine.

The server may be configured to generate a report based on applying the at least one metric to the utilization/billing model associated with at least one of the client identifier; the client organization identifier; and the client location identifier.

In such embodiments the report generated may be based on data log entries for the client only, or similarly the report generated based on the data log entries for a specific organization or sub-division or the organization or the ultrasound machines within a client location or region.

The server may be further configured to analyse the at least one data log to determine at least one metric associated with at least one of the client identifier; the client organization identifier; and the client location identifier.

In such embodiments the analysis may be based on data log entries for the client only, or similarly the analysis performed on the data log entries for a specific organization or sub-division or the organization or the ultrasound machines within a client location or region.

The server may further comprise a monitor configured to monitor the at least one metric, wherein the report generator is configured to generate a report for a client based on the at least one metric monitored.

In such embodiments any significant efficiency or savings may be flagged to the client within a suitable report where a known metric is monitored.

The report generator may comprise a utilization report generator configured to generate a utilization report for a client based on applying the at least one metric to a utilization model.

In such embodiments the efficiency of the operation of the ultrasound machines generating data logs and being analysed can be determined and efficient or non-efficient methods and/or machines indicated to the user.

The report generator may comprise a billing report generator configured to generate a billing report for a client based on applying the at least one metric to a billing model.

In such embodiments the cost efficiency of the operation of the ultrasound machines generating data logs and being analysed can be determined and any cost efficient or economically inefficient methods and/or machines indicated to the user.

The report generator may be further configured to generate workflow instructions to attempt to optimise utilization or workflow associated with the at least one ultrasound machine.

In some embodiments there may be a client comprising: a report request generator configured to generate a utilization/billing request for a server, wherein the utilization/billing request is associated with at least one ultrasound machine; a transceiver configured to transmit the request to the server and further configured to receive a utilization/billing report based on the utilization/billing request.

Thus in some embodiments the client may request utilization and/or billing information in a suitable format to determine whether at least one ultrasound machine is being operated in a cost effective or efficient manner and whether the application of a different utilization and/or billing model would improve the operation of the ultrasound machine.

The client may further comprise a user interface configured to receive at least one of: a client identifier; a client organization identifier; and a client location identifier, the user interface may be configured to be further coupled to the report request generator, wherein the at utilization/billing request may comprise at least one of: the client identifier; the client organization identifier; and the client location identifier when received.

In such a manner the analysis and/or report generation requested by the client can be assisted by enabling the data logs to be requested based on a sub-set of the data logs based on information within the client identifier, the client organization identifier, and the client location identifier.

An ultrasound machine as described herein, may further comprise at least one of: the server as described herein; a database configured to store the at least one data log; and a client as described herein.

An ultrasound resource management system may comprise: at least one ultrasound machine as discussed herein; at least one server as discussed herein; a database configured to store the at least one data log; and at least one client as discussed herein.

In some embodiments there may be provided a method for providing an ultrasound machine comprising: determining at least one utilization data record based on the operation of the ultrasound machine; generating a data log comprising the at least one utilization data record; transmitting the data log to a server configured to analyse the data log of the ultrasound machine.

The method may further comprise generating at least one clock data field, wherein determining at least one utilization data record based on the operation of the ultrasound machine comprises incorporating the at least one clock data field.

The method may further comprise generating at least one operational mode data field, wherein determining at least one utilization data record based on the operation of the ultrasound machine comprises incorporating the at least one operational mode data field.

The method may further comprise generating at least one location data field, wherein determining at least one utilization data record based on the operation of the ultrasound machine comprises incorporating the at least one location data field.

The method may further comprise receiving at least one of: an ultrasound operator identifier; an organization identifier; and a location identifier, wherein determining at least one utilization data record based on the operation of the ultrasound machine comprises incorporating the at least one of: an ultrasound operator identifier; an organization identifier; and a location identifier.

According to a second aspect there is provided a method of providing a server comprising: receiving from at least one ultrasound machine at least one data log comprising at least one data record based on the operation of the ultrasound machine; processing the at least one data log received from the at least one ultrasound machine; storing the at least one data log in a database; analysing the at least one data log to determine at least one metric associated with the at least one ultrasound machine; generating a report for a client based on applying the at least one metric to a utilization/billing model.

Generating a report for a client based on applying the at least one metric to a utilization/billing model may comprise generating a billing report for a client based on applying the at least one metric to the billing model.

The billing model may be a pay-per-use billing model.

Generating a report for a client based on applying the at least one metric to a utilization/billing model may comprise generating the billing report based on at least one of: a charged fixed rate over the length of a contract; a charge based on the initial price and the estimated devaluation of the ultrasound machine due to aging of the technology and/or parts; a charge based on a combination of average duration and number of scans; a charge based on the volume of patients; a charged percentage of national reimbursement tariff for ultrasound scans; a percentage of costs charged to the customer; a charge based on a number of prepaid hours per month at a fixed rate where extra hours have additional cost; a charge based on costs of disposables that are used in procedures; a charge based on a named sonographer; a charge based on a named clinicians; a charge based on a timed rate; a charge for each transducer in use; a charge for each preset of the ultrasound machine in use; a charge based on when the ultrasound machine is powered on; a charge based on the ultrasound machine is used for patient studies; a charge based on the efficiency of usage of the ultrasound machine; a charge based on a number, type and/or length of each study performed by the ultrasound machine; a charge based on the type of department that the scan is required; a charge based on the location of the ultrasound machine; a charge based on the intern& address of the ultrasound machine; a charge based on the time of day; and a charge based on the day of the week.

The processing the at least one data log received from the at least one ultrasound machine may be comprise anonymising named data within the at least one data log before the data is stored on the database.

The method may further comprise receiving at least one request from a client, the request comprising at least one of: a report type identifier; a utilization/billing model identifier; a data log sub-set identifier; a client identifier; a client organization identifier; and a client location identifier.

Generating a report may comprise generating a report based on applying the at least one metric to the utilization/billing model associated with the utilization/billing model identifier.

Generating a report may comprise generating a report based on applying the at least one metric to the utilization/billing model associated with the at least one of: at least one of the client identifier; the client organization identifier; and the client location identifier.

Analysing the at least one data log to determine at least one metric associated with the at least one ultrasound machine may comprise analysing the at least one data log to determine at least one metric associated with at least one of the client identifier; the client organization identifier; and the client location identifier.

The method may comprise monitoring the at least one metric, wherein generating a report is performed based on the at least one metric monitored.

Generating a report may comprise generating a utilization report for a client based on applying the at least one metric to a utilization model.

Generating a report may comprise generating a billing report for a client based on applying the at least one metric to a billing model.

Generating a report for a client based on applying the at least one metric to a utilization/billing model may comprise generating workflow instructions to attempt to optimise utilization or workflow associated with the at least one ultrasound machine.

According to some embodiments there may be provided a method for providing a client comprising: generating a utilization/billing request for a server, wherein the utilization/billing request is associated with at least one ultrasound machine; transmitting the request to the server; and receiving a utilization/billing report based on the utilization/billing request.

The method may further comprise receiving at least one of: a client identifier; a client organization identifier; and a client location identifier, the generating a utilization/billing request for a server further based on at least one of: the client identifier; the client organization identifier; and the client location identifier when received.

The method of providing an ultrasound machine as described herein, may further comprise at least one of: providing the server as described herein; providing a database configured to store the at least one data log; and providing the client as described herein.

A method of providing an ultrasound resource management system may comprise: the method of providing an ultrasonic machine as discussed herein; the method of providing a server as discussed herein; storing the at least one data log; and the method of providing the client as discussed herein.

A non-transitory computer-readable medium containing a computer program may comprise computer program code means adapted to perform all the steps of the methods as discussed herein if the program is run on physical computing device(s).

### BRIEF DESCRIPTION OF THE DRAWINGS

Examples of the invention will now be described in detail with reference to the accompanying drawings, in which:

FIG. 1 shows an example ultrasound resource management system according to some embodiments;

FIG. 2 shows an example ultrasound machine within the ultrasound resource management system shown in FIG. 1 according to some embodiments;

FIG. 3 shows an example server and database within the ultrasound resource management system shown in FIG. 1 according to some embodiments;

FIG. 4 shows an example client within the ultrasound resource management system shown in FIG. 1 according to some embodiments;

FIG. 5 shows a flow chart of the operation of the ultrasound machine within the ultrasound resource management system shown in FIG. 1 according to some embodiments;

FIG. 6 shows a flow chart of the operation of the server for receiving and storing data logs from ultrasound machines within the ultrasound resource management system shown in FIG. 1 according to some embodiments;

FIG. 7 shows a flow chart of the operation of the client and server within the ultrasound resource management system shown in FIG. 1 according to some embodiments;

FIG. 8 shows a flow chart of the operation of the server in generating automated reports within the ultrasound resource management system shown in FIG. 1 according to some embodiments;

FIG. 9 shows an example utilization report chart suitable for including in a report within the ultrasound resource management system shown in FIG. 1 according to some embodiments; and

FIG. 10 shows an example activity chart suitable for including in a report within the ultrasound resource management system shown in FIG. 1 according to some embodiments.

### DETAILED DESCRIPTION OF THE EMBODIMENTS

The concept as embodied in the description herein is an ultrasound resource management system, and the component parts of which being at least one ultrasound machine, a server, a database and a client, configured to enable the generation and analysis of data logs comprising information about the ultrasound machines, the generation of suitable reports such as billing and/or utilization reports to enable the efficient and cost efficient operation of at least one ultrasound machine and in some examples many ultrasound machines which may not necessarily be owned by the same entity.

As discussed herein it would be understood for example that Pay-per-use models when applied to ultrasound machine operations may provide an alternative method of funding capital equipment in environments where budgets do not permit such large capital expenses.

Furthermore in such embodiments as discussed herein by enabling a wide variety of pay-per-use models the operational needs and thus the efficient operation of the ultrasound machines may be met.

In such embodiments as described herein the use of server systems may be distributed globally around the world enables the efficiency and pay-per-use models to be implemented globally.

Furthermore in some embodiments since the equipment is owned by supplier, the supplier can bundle in the service costs into the pay-per-use contract and thus enable the operator to more efficiently operate the machine in terms of being able to determine ‘down’ or ‘servicing’ time is required.

In some embodiments where there is many financing commitments (equipment, facilities, rooms, booking systems etc.) that a more efficient operation of the equipment enables a reduction of the risk associated with the high capital equipment costs of medical imaging equipment.

Furthermore in some embodiments by applying the concepts as discussed herein business models may be constructed that allow a supplier to partner with service providers by taking a percentage of their business, or it's income or profits in exchange for goods.

The generation and collection of utilisation data as discussed herein allows customers to better understand how each individual machine and how groups of machines are being used and make changing to working practices to optimise their efficiency and thus improve the coverage of the machines.

Furthermore it would be understood that in some embodiments any changes in working practices, can be analysed in that comparisons to utilisation metrics and associated charts before and after the change enable the best working practices to be identified and implemented.

The integration of externally sourced data, for example location information can in some embodiments further enable efficient operation practices to be determined as the machines can be moved, showing other patterns of usage which invite further optimisation strategies.

Furthermore in implementing the embodiments described herein the analysis and understanding of the logistics of the operation of the ultrasound machines enables better overall strategies for procurement and usage of equipment for optimal usage.

In some embodiments as described herein the use of a ‘smart’ rule based on the data enables targeted advice to be offered during reporting.

The system as described in further detail herein therefore in some embodiments provides for the secure data collection from ultrasound machines to ‘cloud’ servers, encoded at output of ultrasound machine. This protects the output of the ultrasound machine from being probed for data, keeping it secure from unwanted access. The data is in such embodiments uniquely identifiable to a particular machine and ideally location, where each event is time stamped. The data is in such embodiments uploaded and stored in a ‘cloud’ server in encrypted form and enables access to the information upon request. The data is in some embodiments pre-processed at the server and formed into data structures. These data structures can be searched using commands from a client system, that uploads relevant data from a selected system at selected times and dates for processing. The processing is in some embodiments used to calculate a quote based on a chosen pay-per-use model based on a model of the customer's usage. The data is in some embodiments also represented in a wide range of charts and supporting statistics that characterise the machine's usage, enabling workflow to be visualised, experimented and optimised. This information may in such embodiments be captured in a structured report that is targeted at meeting the needs of different hospital departments, which also has the ability to offer targeted advice.

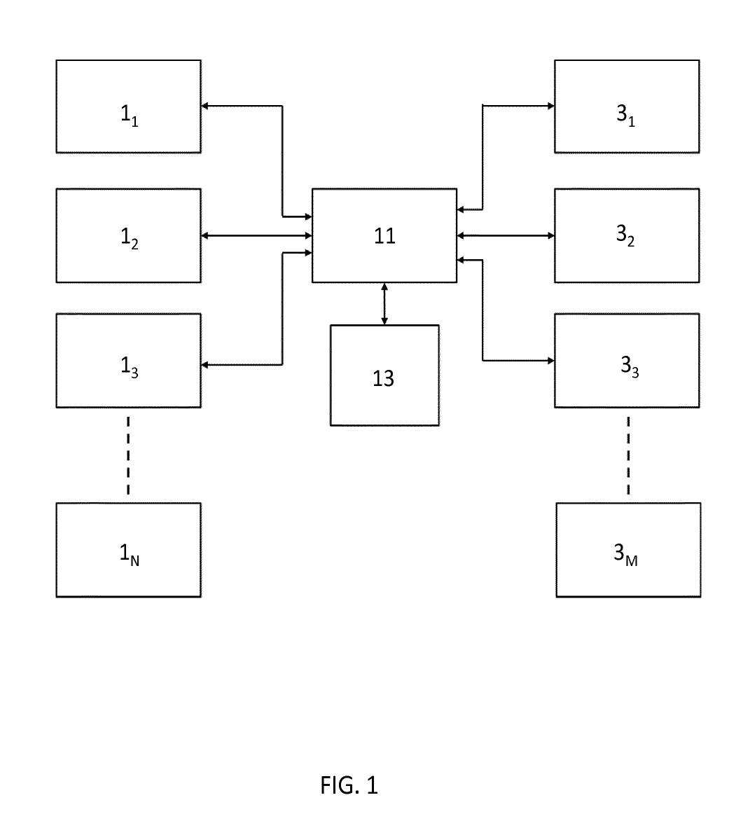

With respect to FIG. 1, an ultrasound resource management system (or an ultrasound utilisation and billing system) is shown.

The ultrasound resource management system comprises at least one ultrasound machine 1. In the example shown in FIG. 1 the ultrasound resource management system comprises N machines which are shown as a first ultrasound machine 1₁, a second ultrasound machine 1₂, a third ultrasound machine 1₃, and a N′th ultrasound machine 1_{N}. It would be understood that the ultrasound machines may be any suitable type of ultrasound machine and may have any suitable range of operations. For example in some embodiments the first ultrasound machine 1₁ is a portable machine, the second ultrasound machine 1₂ a static or fixed location machine, the third ultrasound machine 1₃ a so called 4-D ultrasound machine.

As shown in FIG. 1 the ultrasound machine(s) 1 can in some embodiments be configured to communicate with the server 11 in that the ultrasound machine(s) 1 in some embodiments are configured to transmit data logs to the server 11. The data logs as described in further detail herein comprise operational data with respect the ultrasound machine.

The ultrasound resource management system further comprises a server 11 and a database 13. It would be understood that in some embodiments the server 11 and the database 13 are the same entity or are separate entities. In some embodiments the location of the server 11 and the database 13 is location agnostic. In such embodiments the server 11 can be implemented or exist anywhere on the intern& cloud and connected to any other local servers to store, backup and deliver data (in other words the local servers operate the database functions).

In such embodiments the data logs comprising operational or utilisation data can be collected from many ‘remote’ ultrasound machines by the server and stored in its database.

It would be understood that in some embodiments the data log is first encrypted for transport to a secure data server (on the cloud).

Furthermore it would be understood that in some embodiments the server is configured to pre-process the received data log before storage in the database. The pre-processing of the data log can in some embodiments speed up the access to the data within the data log.

In some embodiments the ultrasound resource management system further comprises at least one client 3 or client device or apparatus. The client(s) 3 as shown in FIG. 1 are shown as M clients 3, which are shown as a first client 3₁, a second client 3₂, a third client 3₃, and a M′th client 3_{M}. The client devices can be any suitable client devices such as any suitable computing apparatus with communications capacity, for example personal computers, tablets, mobile communication equipment and so on. In such embodiments the client(s) can be configured to ‘login’ to the server 11 and browse and access ultrasound data, selecting data from systems at specific dates and times or performing search queries looking for specific data. The server 11 in such embodiments provides a remote or cloud service to allow the data to reside anywhere in the world and allow the data to be accessed anywhere else in the world providing a global solution. In the following examples the client(s) 3 can access the data by requesting a report, such as a utilization or billing report, and receiving a suitable report from the server. However, as discussed herein, any suitable data accessing technique can be employed in some embodiments.

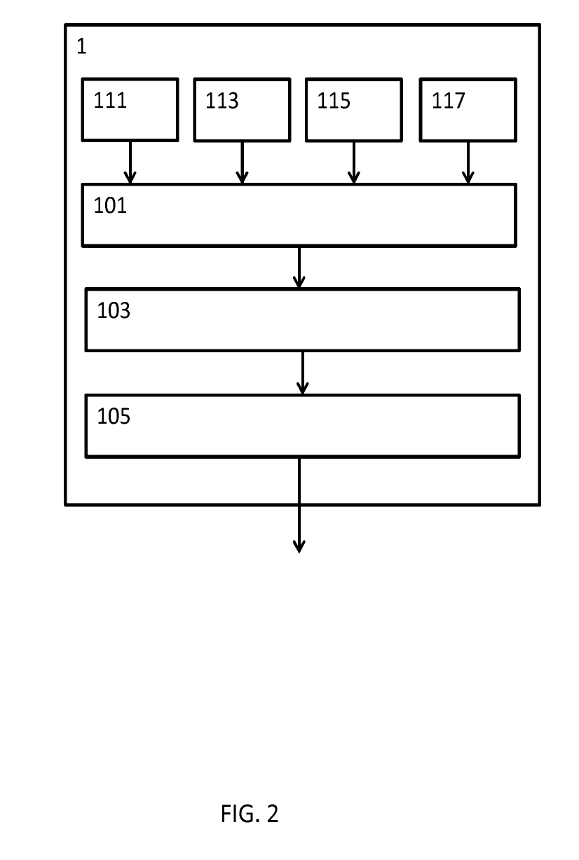

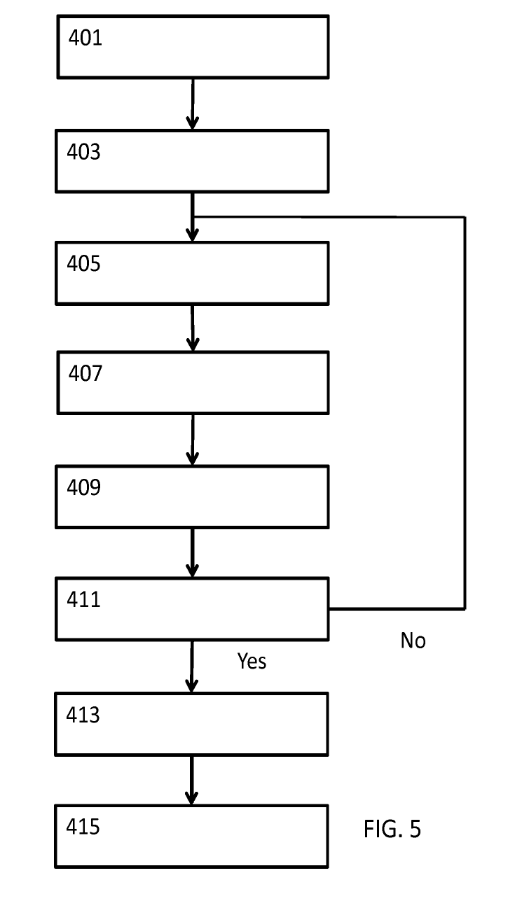

With respect to FIG. 2 an example ultrasound machine 1 is shown in further detail. Furthermore with respect to FIG. 5 the operation of the ultrasound machine 1 according to some embodiments is shown in a flow diagram.

In the following examples the conventional ultrasound parts or components and the operation of the ultrasound machine as typically used are not described and for clarity only the ultrasound machine and the operation of the ultrasound machine with respect to the ultrasound resource management system and the operation of the ultrasound resource management system is described.

In some embodiments the ultrasound machine 1 comprises a utilization determiner 101. The utilization determiner 101 is configured to observe the operation of the machine to determine suitable utilization data records.

Thus for example in order to operate an ultrasound machine it must first be powered on.

The ultrasound machine 1 in some embodiments comprises a user interface 113. The user interface 113 can comprise any suitable user interaction device or apparatus, such as a keyboard, keypad, mouse, trackball, touch screen, switches or other for inputs, a speaker for audio outputs, a display or touch screen display for visual outputs, or any combination thereof.

Thus in some embodiments the operator can switch on the ultrasound machine and the ultrasound machine is initialized.

The operation of switching on/initializing the ultrasound machine is shown in FIG. 5 by step 401.

In some embodiments the ultrasound machine comprises a mode determiner 115 is configured to determine the operational mode of the ultrasound machine. Thus for example in some embodiments the switching on of the ultrasound machine may cause the mode determiner 115 to determine that the ultrasound machine has changed from an ‘off’ mode to an ‘on’ mode or an ‘idle’ mode.

Furthermore in some embodiments the ultrasound machine comprises a clock 111 or timer configured to generate a suitable timing of clock data for the accurate identification of events. Any suitable clock or timer may be employed. For example the clock 111 can in some embodiments generate an initial or first time or clock value when the ultrasound machine is switched on.

This information can then be passed to the utilization determiner 101 which can in some embodiments be configured to generate a first or initial utilization data record. The first or initial utilization data record can thus in some embodiments comprise data fields identifying the first switching on (or first going idle) of the ultrasound machine and the time at which the machine was first switched on (or went idle).

The generation of the first or initial data record is shown in FIG. 5 by step 403.

In some embodiments the user interface may further be configured to enter data or information about the operator of the ultrasound machine, for example entering an operator identifier. The operator identifier can in some embodiments be entered manually or may be performed by any suitable other means or method, such as the operator carrying a suitable tag such as a radio frequency tag comprising the operator identifier information and presenting the tag to a suitable tag reader on or coupled to the ultrasound machine 1.

In some embodiments the user interface may further be configured to enter other data or information about the operator's organization or location. For example in some embodiments the operator can enter an organization identifier. An example of the organization identifier can in some embodiments be the hospital, hospital department, referring GP, AQP or other authorising body. The organisation identifier can in some embodiments be entered manually or may be performed by any suitable other means or method, such as the operator introducing a suitable tag such as a radio frequency tag comprising the organization information and presenting the tag to a suitable tag reader on or coupled to the ultrasound machine 1. Similarly the operator's location can be entered. An example of the location information can in some embodiments be the hospital location, hospital department location, AQP location or other operation location. The location information can in some embodiments be entered manually or may be performed by any suitable other means or method. For example in some embodiments the ultrasound machine can comprise a location determiner 117. The location determiner 117 in some embodiments is configured to use any suitable means for determining the location of the ultrasound machine 1. Any suitable location determination can be performed, for example satellite positioning location determination or radio frequency beacon positioning location determination.

The ultrasound machine 1 may then be operated once a probe (or transducer) is attached to the machine and a software mode is selected. This allows the sonographer use the probe on the patient and creates the diagnostic images and data for the referring Doctor.

In some embodiments the mode determiner 115 can in some embodiments determine the probe type and configuration and generate suitable probe information which is passed to the utilization determiner 101.

Furthermore the mode determiner 115 can be configured to determine when the ultrasound machine 1 is in active operation, in other words performing scanning operations. This mode determination information can then be passed to the utilization determiner 101.

The utilization determiner 101 can in some embodiments having received the information about the mode of operations, timing, and other inputs be configured to generate suitable data records detailing the various operations of the ultrasound machine.

In other words the utilization determiner 101 can in some embodiments be configured to determine an ultrasound machine mode use change.

The operation of determining an ultrasound machine mode use change is shown in FIG. 5 by step 405.

Thus for example when the utilization determiner 101 determines that the ultrasound is being used to perform a scan the utilization determiner 101 can then in some embodiments to generate a suitable utilization data record.

For the example of the ultrasound machine performing a scan the utilization data record can comprise at least one of the following data fields: a mode=active scan field, a time of the scan field, a length of time of scan field, a probe (transducer) identifier field, a software mode field, an operator identifier field, an organization field, a location field. It would be understood that the utilization data record could in some embodiments comprise further suitable fields, for example imaging mode.

The generation of a utilization data record is shown in FIG. 5 by step 407.

The utilization data record can then in some embodiments be passed to a data compiler 103.

In some embodiments the ultrasound machine 1 further comprises a data compiler 103. The data compiler 103 is in some embodiments configured to receive the utilization data records from the utilization determiner 101 and generate a suitable data log or data log file for transmission to the server 11.

Thus in some embodiments the data compiler 103 on receiving the utilization data record, is configured to append it to a data log.

The operation of appending the utilization data record to the data log is shown in FIG. 5 by step 409.

In some embodiments the data compiler is further configured to determine or check whether the appended data log is to be uploaded to the server 11. It would be understood that in some embodiments the uploading of the data log is performed on a near real time basis, in other words after every new generated record. However it would be understood that in some embodiments the log is compiled over a period of time, for example a couple of hours, a working day, a week or other defined time and the log file uploaded once the time period is ended.

The operation of determined whether the data log is to be uploaded is shown in FIG. 5 by step 411.

In the example shown in FIG. 5, where it is determined not to upload the data log, the operation passes back to step 405 where the utilization determiner 101 is observing the operation of the ultrasound machine 1 and waits to determine a change in the use of the ultrasound machine.

Where it is determined to upload the data log the data compiler 103 is further configured in some embodiments to encrypt the data log to prevent unauthorised access to the data. The encryption can be performed according to any suitable encryption mechanism. For example in some embodiments the ultrasound machine 1 and the server 11 agree a shared secret which is used to generate a suitable encryption key. In other embodiments the server 11 is configured to pass to the ultrasound machine 1 a public key to be used to encrypt the data log which can then be decrypted using the server 11 private key. In the example shown herein the encryption is performed to the data log as a whole, however it would be understood that in some embodiments the encryption can be performed on a record by record basis as the record is appended to the data log.

The operation of encrypting the data log is shown in FIG. 5 by step 413.

The data compiler 103 can then in some embodiments be configured to pass the data log (which in some embodiments is encrypted or comprises encrypted data records) to the transmitter 105.

In some embodiments the ultrasound machine 1 comprises a transmitter 105. The transmitter 105 can comprise any suitable transmitter apparatus and in some embodiments is part of a transceiver apparatus. The transmitter 105 is configured to process the data log and transmit it according to any suitable means to the server 11. Thus in some embodiments the transmitter is a suitable wireless transmitter (such as an IEEE802.11 or similar WiFi transmitter, or cellular transmitter) configured to communicate to the server via a suitable access point and via the internet to the server 11 operating ‘located’ within the ‘cloud’. It would be understood that in some embodiments the transmitter is a suitable wired transmitter.

The operation of transmitting the data log to the server is shown in FIG. 5 by step 415.

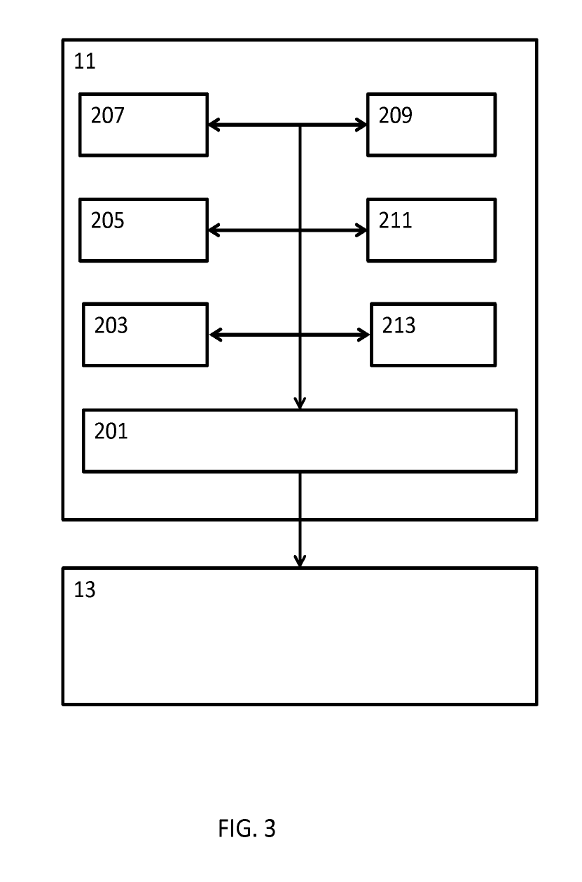

With respect to FIG. 3 an example of a server 11 and database 13 is shown with respect to some embodiments. Furthermore with respect to FIGS. 5 to 8 various operations of the server 11 and database 13 are shown in flow diagram form.

In some embodiments the server 11 comprises a transceiver 201. The transceiver 201 in some embodiments is configured to interface the server 11 with any external device or apparatus. Thus for example the transceiver 201 can be configured to handle communication with the ultrasound machines 1, the database 13, and the clients 3. It would be understood that in some embodiments the transceiver 201 can be separated into transmitter and receiver parts, and furthermore in some embodiments the transceiver 201 comprises different parts or differently configurable parts for communicating with different entities. For example the transceiver 201 can in some embodiments comprise a first transceiver configured to communicate with the ultrasound machines 1, a second transceiver configured to communicate with the database 13 and a third transceiver configured to communicate with the client(s) 3.

The transceiver 201 in some embodiments can be configured to receive the data log(s) from at least one ultrasound machine. It would be understood that in some embodiments the server 11 and therefore the transceiver 201 is configured to receive the data logs from a number or many ultrasound machines.

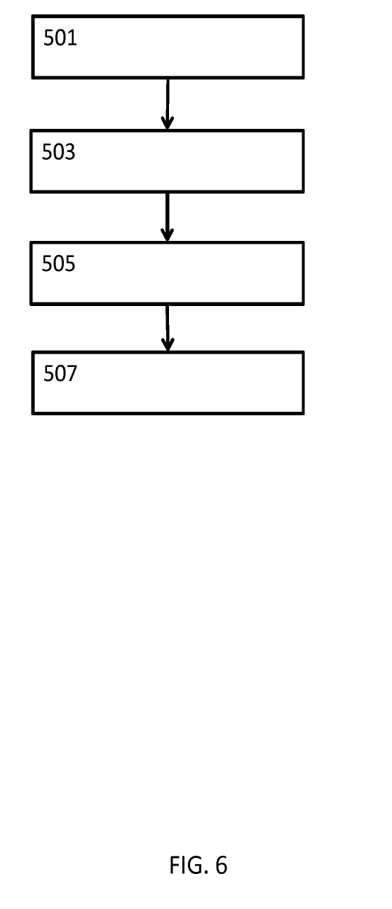

The operation of receiving the data log from at least one ultrasound machine 1 is shown in FIG. 6 by step 501.

In some embodiments the transceiver 201 is configured to pass the data log from the at least one ultrasound machine 1 to a record parser/processor 203.

In some embodiments the server 11 comprises a record parser/processor 203. The record parser/processor 203 is in some embodiments configured to receive the data log and perform processing and/or parsing of the data log to generate a suitable data format for storage.

For example in some embodiments the record parser/processor 203 is configured to decrypt the data log and/or the data records within the data log.

The operation of decrypting the data log is shown in FIG. 6 by step 503.

Furthermore an example of processing the data log is one of adding an additional field identifying the ultrasound machine 1 from which the data log was received. For example in some embodiments the intern& protocol (IP) address or the media access control (MAC) address associated with the ultrasound machine is appended to the data log and the utilization data records.

In some embodiments the record parser/processor 203 can process the data records to remove redundant or duplicate utilization data records from the data log.

In some embodiments the record parser/processor 203 can be configured to anonymise data such as names, for example the referring GP or sonographer, before the data is stored on the database 13. Thus in some embodiments a name is associated with an ID label or number in such a way that they are traceable as an individual but not named.

Furthermore in some embodiments the record parser/processor 203 can be configured to analyse and process the data log and the utilization data records within the data log so that the data is more easily accessed by further analysis and report generation operations.

The operation of processing/analysing the data log is shown in FIG. 6 by step 505.

The data log information is then in some embodiments passed to the database 13 for storage. The database as described herein can be any suitable means or apparatus configured to store the data log or the processed data log information in a suitable format.

The operation of storing the data log in the database is shown in FIG. 6 by step 507.

The server 11 in some embodiments comprises a record analyser 205. The record analyser 205 in some embodiments is configured to analyse the data log information stored in the database 13 and the utilization data records within the data log to generate at least one suitable metric value for describing the operation of the at least one ultrasound machine 1 over a period of time.

For example the record analyser 205 can in some embodiments be configured to generate at least one metric from the following list: Elapsed time/days active; ultrasound machine powered-on time; ultrasound machine In-use time; ultrasound machine Idle time ultrasound machine Efficiency of utilisation (defined as in-use time as a percentage of powered-on time); ultrasound machine Number/identifier; department of referring Doctors/organization; Number of sonographers or operators; Number and type of studies/scans; Length or average duration of studies/scans; Time for each transducer/probe in-use; Time for each preset (software mode) in-use. However it would be understood that any suitable metric can be generated.

This information can in some embodiments be passed to a report generator such as a billing report generator 213 and/or the utilization report generator 211.

In some embodiments the server comprises a utilization report generator 211.

The utilization report generator 211 in some embodiments is configured to receive at least one metric from the record analyser. The utilization report generator 211 can then in some embodiments generate a utilization report based on applying a determined utilization model to the metric.

The utilization model can be in some embodiments a filtering or processing of the metrics generated from the at least one ultrasound machine representing the operational efficiency or utilization of the at least one ultrasound machine. In some embodiments the utilization model determines certain combinations of graphs and metrics which can be used to accurately characterise a machine. These can be brought together to specifically facilitate the analysis by specific stakeholders of the at least one ultrasound machine (such as for example various departments of a hospital which ‘own’ the ultrasound machine.

In some embodiments the generation of the report comprises the generation of ‘Dashboards’ that are customised to a viewer's need. However it would be understood that in some embodiments the report generation is one which attempts to improve the utilization of the at least one ultrasound machine based on the metric.

As typically healthcare organisations such as hospitals have many departments and roles within the organisation allowing them to focus on activities within their control. The utilization report generator 211 can be configured to generate a report such that it provides utilization data or information suitable with respect to the local factors that influence the productivity and utilisation of the staff and resources.

An example of this is where the utilization report generator 211 is configured to meet the needs of 4 different departments heads within a typical hospital in which all have a slightly different focus. These can, for example, be a department superintendent who is in charge of sonographers, and would be specifically interested in which transducers were used and for how long. There can also be a department manager who would be in charge of specialist department (e.g. radiology), and would be specifically interested in which doctors and consultants use the machine, possibly to bill them for usage. Furthermore the hospital may have a finance director who is in charge of hospital procurement, and would be specifically interested in whether their ultrasound machines are used to their full potential, what additional machines are required and their specification, the cost and billing of machines. Also there may be an IT manager, who is in charge of computers & networks, and would be specifically interested in the utilisation of machines and managing the headcount of staff.

Therefore in some embodiment the utilization model selected is based on the identity of the client in order that the generated utilisation report meets each of these department needs.

The utilization report generator 211 in some embodiments by applying the determined utilization model to the at least one metric can be configured to determine potential issues with respect to the utilization of the at least one ultrasound machine and thus generate new workflow instructions to attempt to optimise utilization or workflow associated with the at least one ultrasound machine.

Thus for example the utilization report generator 211 can in some embodiments identify daily patterns, for example start of day power-up and test, preparations before the first patient and length of working day. The utilization report generator can further in some embodiments identify breaks in activity, for example lunch breaks, times between patients, set-up time and time at day end. The utilization report generator 211 can further in some embodiments analyse weekend usage and out of normal working hours, realising the opportunities and available resources when machine is not usually used. The utilization report generator 211 can further in some embodiments track the location of the ultrasound machine and therefore understand where the machine is during the working week, understand how the machine is being used when not under the normal department jurisdiction.

Furthermore example of changing working practices can include the utilization report generator controlling or changing the time between patient appointments, changing the times when the first or last patients studies can begin, changing patient queuing times, Re-tasking the machine during parts of the day, or week to another department when there is normally a low load (e.g. weekends).

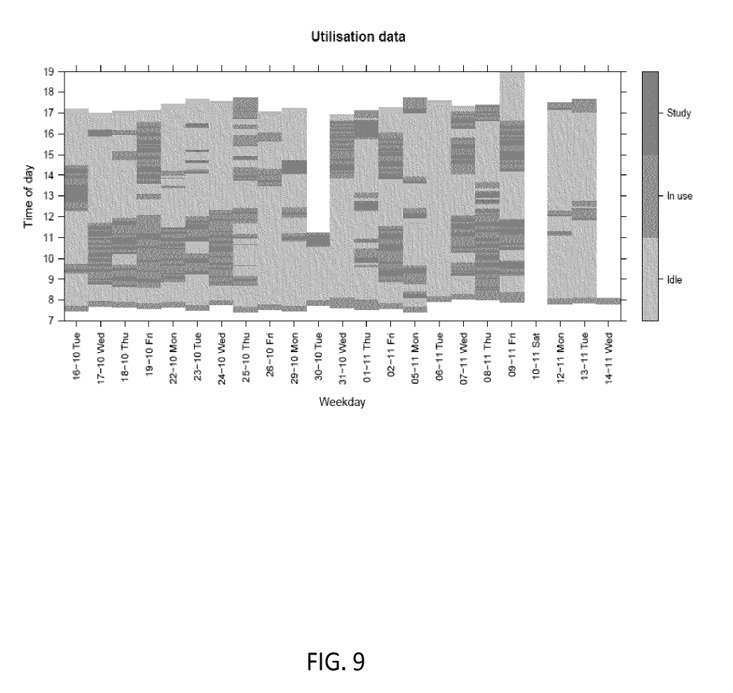

A graphical example of the output of the utilization report generator is shown in FIG. 9 which shows a chart showing an example utilisation of an ultrasound system over the elapsed 1 month timeframe. The x-axis as shown in FIG. 9 shows the weekday and date, the y-axis in FIG. 9 shows the time of day, where the dark shaded data refers times at which the machine is running a study with a patient, is otherwise in-use in medium shading or idle in light shading. The data in this example indicates plenty of scope for an increase in patient throughput; at the start of the day, during lunchtime each day and at other times of the day and at weekends.

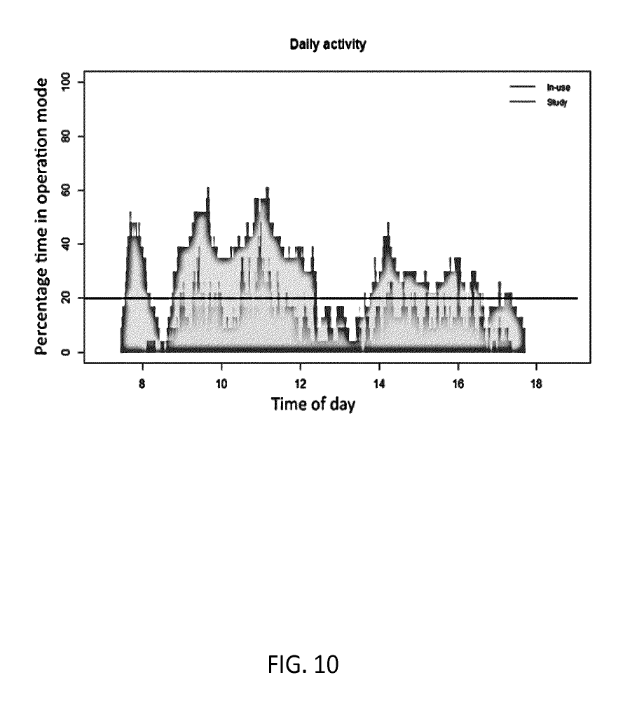

A further example of the output of the utilization report generator is shown in FIG. 10 which shows a chart showing an example utilisation based on the same 1 month data as shown in FIG. 9 set to show the daily utilisation mode at any given time of day. In this example the time of day on the x-axis and the y-axis shows what percentage of the time the machine is being used for a patient study (dark shading) or in-use (light shading), where studies are considered as part of the in-use time.

This figure shows a pattern of behaviour, which can be derived (by thresholding the top 20 percent of time in operation mode) to reveal: Normal working times from 07:32 to 17:23 lasting 9 hours 51 minutes. First patient study begins at 08:06 Also there is often a lull in activity at: 08:13 lasting 31 minutes, 12:26 lasting 71 minutes, 16:39 lasting 22 minutes. The chart also indicates a periodicity in the data (showing clear peaks) to indicate times where the machine is at its busiest: 07:50, 09:39, 11:08 and 14:13 and that the machine is clearly busier in the morning than in the afternoon. This analysis provides options to provide targeted advice in reporting but also to enable the utilization report generator to effect control of the booking of future ultrasound appointments to fill the underutilized periods.

In some embodiments the server 11 comprises a billing report generator 213 configured to receive the metrics from the record analyser and configured to generate a report for a client based on applying the at least one metric to a billing model.

In such embodiments the billing report generator 213 can determine and apply a suitable billing model, for example a determined pay-per-use billing model for which the metric, such as the quantity measured can be combined with a business cost rate to calculate an itemised bill (typically for each month). In such embodiments the billing report generator 213 can determine a bill determining a charged fixed rate over the length of a contract. However it would be understood that the billing report generator 213 can in some embodiments determine a bill determining a charge based on the initial price and the estimated devaluation of the machine due to aging of the technology and/or parts, or a charge based on a combination of average duration and number of scans, or a charge based on the volume of patients, or a charged percentage of national (NHS) reimbursement tariff for ultrasound scans, or a percentage of costs charged to the customer, or a charge based on a number of prepaid hours per month at a fixed rate where extra hours have additional cost, or a charge based on costs of disposables that are used in procedures, or a charge based on the named sonographers, or based on named clinicians (where list of names are agreed in contract, or a number per department). In some embodiments the billing report generator 213 can in some embodiments determine a bill determining a charge based on a timed rate: for each transducer is in use; for each preset is in use; that device is powered on; that the device is used for patient studies; on the efficiency of usage; for the number, type and/or length of each study; for the type of department that the scan is required; for the location of the machine; for the intern& address of the machine; of the time of day; of the day of the week or any combination of the above.

For example the billing report generator 213 may be configured to use a combination of a fixed month charge plus additional charge based on the usage of transducers. In such a situation where 6 transducers are supplied, but 2 are assigned as standard transducers, the usage time of the non-standard transducer is recorded and charged at an additional hourly rate. This can cause in some embodiments the billing report generator 213 to determine a billing model comprising

Monthly standing costs: £1800

Hourly rate for non-standard transducers: £35

The metrics from the utilisation data of the machine record:

Number of months: 1

Time for non-standard transducers: 21.6 hours

The billing report generator 213 can then for example generate a bill using the following expression

total income=monthly cost+(additional hourly rate*number of hours)

total income=1800+(35*21.6)=£2556

The billing report can then be passed via the transmitter to the client requesting it or to an addressed client.

In some embodiments the billing report generator 213 can be configured to generate an ‘itemised bill’. For example a report is generated itemising the cost for each scan or each department, each location or each referring physician.

In some embodiment the server 11 comprises a utilization monitor 209. The utilization monitor 209 in some embodiments is configured to monitor a determined or defined metric produced by the record analyser 205.

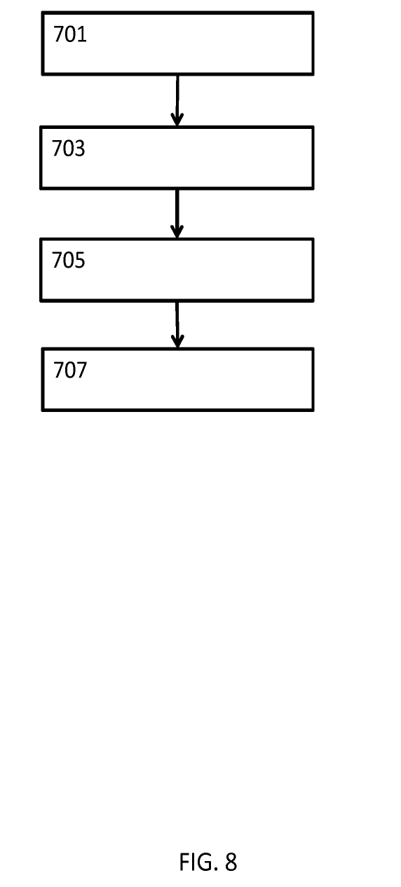

The operation of monitoring the data log for a specific metric is shown in FIG. 8 by step 701.

The utilization monitor 209 can in some embodiments compare the determined or defined metric (or in some embodiments a combination of metrics) against a determined or defined threshold value, such as a billing or utilization threshold value. For example in some embodiments the metric is percentage utilization and the threshold value is 10%. Where the metric passes the threshold value then the utilization monitor 209 can be configured to trigger or request that a report be generated by an associated report generator.

The operation of determining when the threshold value is passed based on the metric value is shown in FIG. 8 by step 703.

The associated report generator, such as the utilization report generator 211 and/or the billing report generator 213 is configured then to generate the report based on the request. For example in the situation of low utilization the utilization report generator 211 can generate a report to determine whether the ultrasound machine 1 could be better allocated.

The operation of generating the report is shown in FIG. 8 by step 705.

The report could then be transmitted or passed to a suitable means for further processing and/analysis such as for example in the case of low utilization a central booking facility to allocate any overflow from other ultrasound machines in the area or at the hospital.

The transmission of the report is shown in FIG. 8 by step 707.

An automatically generated report in such embodiments is more useful still when it can also provide appropriately targeted advice. It would be understood that rule for triggering advice to a report also needs to be carefully judged and in some embodiments clearly separated from the factual text, graphs and numbers that are otherwise presented in some reports. A simple example is if the machine efficiency (time in use/time machine is powered on) is <60%. In this case an advice rule could fire that adds text to the report suggesting that benchmark systems operate at around 70% efficiency, which is ideal. Such a rule could then trigger yet another analysis rule to examine the typical working day and highlight the areas, for example where there is a lull in activity, where changes to working practices are possible. A cascade of advice can therefore present options for improvement as one rule fires another to create targeted advice based on the available data. This advice can be separated from the normal report by creating a structural element, e.g. text in a box with a background colour—so that the viewer will separate advice or conclusions from the factual data presented in the report. Further, the colour of the box could indicate the role of the clinical professional that the advice is targeted at. Alternatively different reports, or sections of a report could be formulated for different departments, containing only the statistics, graphs and advice relevant to their department.

In some embodiments the server further comprises a request determiner 207. The request determiner is configured to receive and process requests for reports from client(s) 3.

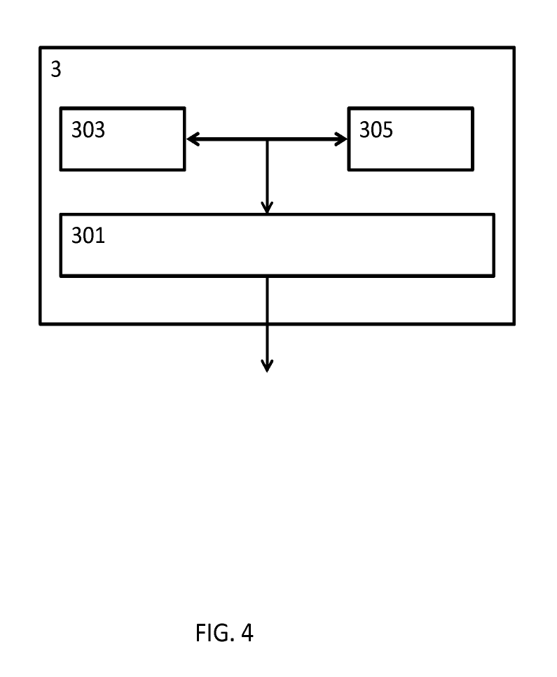

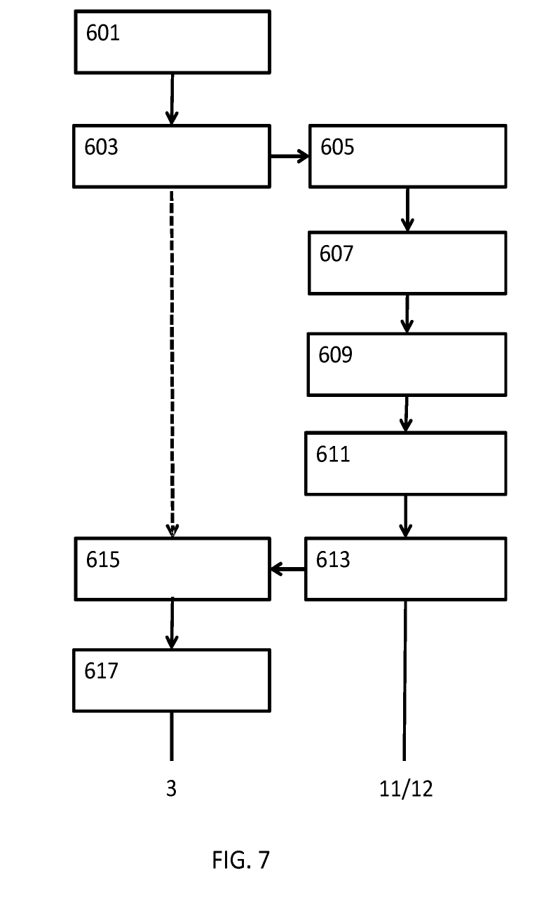

With respect to FIG. 4 an example client 3 or client apparatus or device is shown. Furthermore an example operation of the client shown in FIG. 4 when interacting with the server 11 and database shown in FIG. 3 is shown in FIG. 7.

The client 3 in some embodiments comprises a user interface 305. The user interface 305 can comprise any suitable user interaction device or apparatus, such as a keyboard, keypad, mouse, trackball, touch screen, switches or other for inputs, a speaker for audio outputs, a display or touch screen display for visual outputs, or any combination thereof.

In some embodiments the user interface 305 can be configured to enter data or information about the user or operator of the client, for example entering a client identifier. The client identifier can in some embodiments be entered manually (such as by performing a log on process, or by entering a user identifier as part of the information recorded for a patient scan) or may be performed by any suitable other means or method, such as the operator carrying a suitable tag such as a radio frequency tag comprising the client identifier information and presenting the tag to a suitable tag reader on or coupled to the client apparatus 3.

In some embodiments the user interface 305 may further be configured to enter other data or information about the client's organization or location. For example in some embodiments the client can enter an organization identifier. An example of the client organization identifier can in some embodiments be the hospital, hospital department, referring GP, AQP or other authorising body. The client organisation identifier can in some embodiments be entered manually or may be performed by any suitable other means or method, such as the client introducing a suitable tag such as a radio frequency tag comprising the client organization information and presenting the tag to a suitable tag reader on or coupled to the client apparatus 3. Similarly the client's location (or desired analysis or reporting location) can be entered. An example of the client location information can in some embodiments be the hospital location, hospital department location, AQP location or other operation location. The client location information can in some embodiments be entered manually or may be performed by any suitable other means or method. For example any suitable means for determining the location of the client apparatus 3, for example satellite positioning location determination or radio frequency beacon positioning location determination, IP address determination, or WiFi positioning determination.

Furthermore in some embodiments the client or user can be configured to use the user interface to request reporting data about at least one ultrasound machine. However it would be understood that in some embodiments the generation of the report request is an automatic or semi-automatic one, for example triggered by a booking system attempting to optimise the utilization of the ultrasound machines available to it, or triggered at specific times of the day, week, month or year.

The client in some embodiments further comprises a request generator 303 configured to generate a report request. In some embodiments the request generator 303 is configured to generate the request comprising a report type identifier, for example whether a billing report or a utilization report is requested. The report type identifier can in some embodiments be determined by the request generator 303 based on information on the client. For example the role or operation of the requester.

In some embodiments the request generator 303 is configured to generate the request comprising a utilization/billing model identifier, for example indicating what model (billing or utilization model) is to be applied.

In some embodiments the request generator 303 is configured to generate the request comprising a data log sub-set identifier, for example generating utilization reports for a specific time of the day or day of the week, such that the workflow at that time/day can be controlled more efficiently.

In some embodiments the request generator 303 is configured to generate the request comprising a client identifier, for example enabling a filtering of the metrics generated or a filtering of the data prior to generating the metric to reflect only the ultrasound machines ‘responsible to’ or ‘belonging to’ or ‘operated by’ a client matching that identity.

In some embodiments the request generator 303 is configured to generate the request comprising a client organization identifier, for example enabling a filtering of the metrics generated or a filtering of the data prior to generating the metric to reflect only the ultrasound machines ‘responsible to’ or ‘belonging to’ or ‘operated by’ a that organisation or a related organisation.

In some embodiments the request generator 303 is configured to generate the request comprising a client location identifier, for example enabling a filtering of the metrics generated or a filtering of the data prior to generating the metric to reflect only the ultrasound machines within a nearby or neighbouring region to the region or location identified.

The operation of generating the (utilization/billing) report request is shown in FIG. 7 by step 601.

The client 3 in some embodiments further comprises a transceiver 301. The transceiver 301 can comprise any suitable transmitter apparatus and receiver apparatus. The transceiver 301 is configured to receive the report request and transmit it according to any suitable means to the server 11. Thus in some embodiments the transceiver is a suitable wireless transceiver (such as an IEEE802.11 or similar WiFi, or cellular transceiver) configured to communicate to the server via a suitable access point and via the intern& to the server 11 operating ‘located’ within the ‘cloud’. It would be understood that in some embodiments the transceiver is a suitable wired transceiver.

The operation of transmitting the request to the server 11 is shown in FIG. 7 by step 603.

The server 11 and in some embodiments the server transceiver 201 receives the request from the client 3 or client apparatus 3.

The operation of receiving the request from the client 3 is shown in FIG. 7 by step 605.

The server transceiver 201 in some embodiments passes the request to the request determiner 207. The request determiner 207 is then configured to analyse the request. The analysis of the request can in some embodiments lead to the request determiner controlling the operation of retrieving data logs comprising utilization records, analysis of the retrieved data logs to generate or determine at least one suitable metric value(s) and the generation of the requested report by the associated report generator.

The operation of analysing the request is shown in FIG. 7 by step 607.

The record analyser 205 can as described herein based on the request analysis determine data to be retrieved from the database and furthermore determine at least one metric value(s) based on the data retrieved from the database. In some embodiments where pre-processing of the data has been performed the pre-processed metric values can be retrieved from the database.

The retrieval of the data logs based on the request analysis is shown in FIG. 7 by step 609.

The analysis of the data logs to generate at least one metric based on the request analysis is shown in FIG. 7 by step 611.

The report generator, such as the utilization report generator 211 and/or the billing report generator 213 can then in some embodiments generate at least one report based on the at least one determined metric values or a combination of determined metrics values when applied to the determined report model. In some embodiments the report model is itself determined based on the request analysis (for example a utilization report of an ultrasound machine is generated for the booking client).

The operation of generating a report based on the application of a utilization/billing model to the at least one determined metric values is shown in FIG. 7 by step 613.

The report can then in some embodiments be passed to the server transceiver 201, which then transmits the report to the client transceiver 301.

The operation of transmitting the report from the server 11 to the client 3 is shown in FIG. 7 by step 615.

The client transceiver 301 can then in some embodiments be configured to receive the report and pass the report onto a suitable means for processing/display.

The operation of receiving the report at the client 3 is shown in FIG. 7 by step 617.

It would be understood that although in the above examples the ultrasound machine 1, the server 11, database 13, and client 3 are shown as physically separate or distant devices, that in some embodiments the functionality or at least some of the functionality can be implemented within a single device. For example in some embodiments the ultrasound machine 1 can be configured to implement at least some of the functionality of the server 11 and database 13 in determining, storing and processing utilization and/or billing data, and furthermore in some embodiments be configured to implement at least some of the functionality of the client 3 in that in some embodiments utilization and/or billing data can be displayed to the user of the ultrasound machine 1. For example where an external connection is not available as the ultrasound machine 1 is operating in an environment with poor communication capacity either designed for (for example within a faraday cage) or actual (for example within a room with no windows in an old thick walled building with poor communication distribution). In such embodiments the transceiver/transmitters implemented within the ultrasound machine 1, the server 11 and the client 3 functions can be considered to be internal communications between modules or functions of the device.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. The embodiments may be implemented by means of hardware comprising several distinct elements. In the device claim enumerating several means, several of these means may be embodied by one and the same item of hardware. The mere fact that certain measures are recited in mutually different dependent claims does not indicate that a combination of these measures cannot be used to advantage. Furthermore in the appended claims lists comprising “at least one of: A; B; and C” should be interpreted as (A and/or B) and/or C.

1. A server comprising:
a transceiver configured to receive from each of a set of ultrasound machines at least one data log comprising at least one data record based on the operation of the ultrasound machine;
a record parser configured to process the data logs received from the set of ultrasound machines before storing the data logs in a database;
a record analyser configured to analyse the data logs to determine at least one metric associated with each ultrasound machine;
a report generator configured to generate a report for a client based on applying the metrics to a utilization/billing model.

2. The server as claimed in claim 1, wherein the report generator comprises a billing report generator configured to generate a billing report for a client based on applying the metrics to the billing model.

3. The server as claimed in claim 2, wherein the billing model is a pay-per-use billing model.

4. The server as claimed in claim 2, wherein the billing report generator configured to generate a billing report for a client based on applying the at least one metric to a billing model is configured to generate the billing report based on at least one of:
a charged fixed rate over the length of a contract;
a charge based on the initial price and the estimated devaluation of the ultrasound machine due to aging of the technology and/or parts;
a charge based on a combination of average duration and number of scans;
a charge based on the volume of patients;
a charged percentage of national reimbursement tariff for ultrasound scans;
a percentage of costs charged to the customer;
a charge based on a number of prepaid hours per month at a fixed rate where extra hours have additional cost;
a charge based on costs of disposables that are used in procedures;
a charge based on a named sonographer;
a charge based on a named clinicians;
a charge based on a timed rate;
a charge for each transducer in use;
a charge for each preset of the ultrasound machine in use;
a charge based on when the ultrasound machine is powered on;
a charge based on the ultrasound machine is used for patient studies;
a charge based on the efficiency of usage of the ultrasound machine;
a charge based on a number, type and/or length of each study performed by the ultrasound machine;
a charge based on the type of department that the scan is required;
a charge based on the location of the ultrasound machine;
a charge based on the internet address of the ultrasound machine;
a charge based on the time of day; and
a charge based on the day of the week.

5. The server as claimed in claim 1, further comprising a request determiner configured to receive at least one request from a client, the request comprising at least one of:
a report type identifier;
a utilization/billing model identifier;
a data log sub-set identifier;
a client identifier;
a client organization identifier; and
a client location identifier.

6. The server as claimed in claim 5, where the request comprises the utilization/billing model identifier, wherein the report generator is further configured to generate a report based on applying the at least one metric to the utilization/billing model associated with the utilization/billing model identifier.

7. The server as claimed in claim 5, where the request comprises at least one of the client identifier; the client organization identifier; and the client location identifier, wherein the report generator is further configured to generate a report based on applying the at least one metric to the utilization/billing model associated with at least one of the client identifier; the client organization identifier; and the client location identifier.

8. The server as claimed in claim 7, where the request comprises at least one of the client identifier; the client organization identifier; and the client location identifier, wherein the record analyser is further configured to analyse the at least one data log to determine at least one metric associated with at least one of the client identifier; the client organization identifier; and the client location identifier.

9. The server as claimed in claim 8, further comprising a monitor configured to monitor the at least one metric, wherein the report generator is configured to generate a report for a client based on the at least one metric monitored.

10. The server as claimed in claim 9, wherein the report generator is further configured to generate workflow instructions to attempt to optimise utilization or workflow associated with the at least one ultrasound machine.

11. A method for providing a server comprising:
receiving from each of a set of ultrasound machines at least one data log comprising at least one data record based on the operation of the ultrasound machine;
processing the logs received from the set of ultrasound machines;
storing the data logs in a database;
analysing the data logs to determine at least one metric associated with each ultrasound machine;
generating a report for a client based on applying the metrics to a utilization/billing model.

12. The method as claimed in claim 11, wherein generating a report comprises generating a billing report for a client based on applying the at least one metric to the billing model.

13. The method as claimed in claim 12, wherein the billing model is a pay-per-use billing model.

14. The method as claimed in claim 13, further comprising monitoring the at least one metric, wherein the generating a report for a client further comprises generating a report for a client based on the at least one metric monitored.

15. The method as claimed in claim 14, wherein generating a report for a client based on applying the at least one metric to a utilization/billing model further comprises generating workflow instructions to attempt to optimise utilization or workflow associated with the at least one ultrasound machine.
